(19) United States
(12) Patent Application Publication
(10) Pub. No.: US 2006/0058364 A1
(43) Pub. Date: Mar. 16, 2006

(54) Treatment of dependence and dependence related withdrawal symptoms

(76) Inventors: Antti Haapalinna, Turku (FI); Timo Viitamaa, Turku (FI); Raimo Virtanen, Rusko (FI)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/509,152

(22) PCT Filed: Mar. 28, 2003
(86) PCT No.: PCT/FI03/00240

§ 371 Date: Oct. 14, 2005

Related U.S. Application Data
(60) Provisional application No. 60/368,165, filed on Mar. 29, 2002.

(51) Int. Cl. A61K 31/4164

(52) U.S. Cl. 514/396

(57) ABSTRACT

A method for treatment of dependence and dependence related withdrawal symptoms caused by the discontinuation of subacute or chronic use of psychostimulant agents, to ease a patient's withdrawal from the psychostimulants with an alpha2-adrenoceptor antagonist or a pharmaceutically acceptable ester or salt thereof.

### FIELD OF THE INVENTION

The present invention relates to a method of the treatment of dependence and dependence-related withdrawal symptoms caused by the discontinuation of the use of psychostimulant drugs. The present invention relates to the use of selective alpha2-adrenoceptor antagonists in the treatment of dependence and said symptoms and how the compounds can be used generally to ease a patient's withdrawal from psychostimulants.

### BACKGROUND OF THE INVENTION

Clinically the current treatment strategy has been to use a drug of the same drug class as the drug that caused the dependence and withdrawal symptoms after discontinuation of the use, e.g. methadone and buprenorphine in morphine, heroin, meperine, etc. withdrawal. The dose of the substituting drug is then decreased gradually in order to prevent too massive withdrawal symptoms. This has been somewhat problematic, because the substituting drugs are usually also addictive and classified as narcotics and after discontinuation of the substituting drug there usually are withdrawal symptoms. Also the relapses to use the addictive drug are very common by using this treatment strategy.

Dopamine is a neurotransmitter that influences many functions and has effects on motor control, cognitive and emotional functions. The dopaminergic system is disrupted in various neuropsychiatric disorders and conditions such as Parkinson's Disease, schizophrenia, aggressive behavior, anhedonia etc. Psychostimulants like amphetamine and cocaine enhance dopamine release and inhibit dopamine uptake from the synaptic cleft in the CNS. This phenomenon is generally associated with abuse liability of psychostimulant agents and with the development of drug dependency, caused by subacute and/or chronic use of the psychostimulant agents.

The drug discrimination (generalization) approach has been widely utilized to determine if a drug-induced stimulus will substitute for other drugs of a specific class and is a widely used method in studies on central effects of various psychogenic drugs. An animal trained to discriminate a dose of a particular (possibly hallucinogenic) agent will display stimulus generalization (substitute) only to agents having a similar kind of net effect, although not necessarily of a totally identical mechanism of action (Cunningham K. A. and Appel J.P.Discriminative stimulus properties of cocaine and phencyclidine: similarities in the mechanism in the action. pp. 181-192. In Colpaert, F. C., Slangen, J. L. (eds.) Drug discrimination: Applications in CNS pharmacology, Elsevier Biomedical Press, Amsterdam, 1982). Thus, it is suggested that a drug-appropriate response with a tested drug is some function of the proportion of pharmacological effects in the test set associated with pharmacological net effects of the generalized drug i.e. reinforcement during drug discrimination training. When the training drug is generalized only partially, it is suggested that there are common pharmacological effects, but the overlap of the net effects of the training drug and challenge drug is only partial (Glennon, R. A., Rosencrans J. A., Young, R. The use of the drug discrimination paradigm for studying hallucinogenic agents. A review, pp. 69-96. In Colpaert, F. C., Slangen, J. L. (eds.) Drug discrimination: Applications in CNS pharmacology, Elsevier Biomedical Press, Amsterdam, 1982; Stolerman, I., Mello, G. Role of training conditions in discrimination of central nervous system stimulants by rats. Psychopharmacology 73: 295-303, 1981).

It has been previously published that dopaminergic agents (dopamine agonists and dopamine uptake inhibitors) are generalised to psychostimulants such as amphetamine and cocaine, but noradrenaline uptake inhibitor desipramine only weakly (Stolerman, I., Mello, G. Role of training conditions in discrimination of central nervous system stimulants by rats. Psychopharmacology 73: 295-303, 1981; Porsolt R. D., Pawelec C. and Jalfre M. Use of a drug discrimination procedure to detect amphetamine-like effects of antidepressants. pp. 193-202. In Colpaert, F. C., Slangen, J. L. (eds.) Drug discrimination: Applications in CNS pharmacology, Elsevier Biomedical Press, Amsterdam, 1982). Thus, the cue properties of psychostimulants are mediated through dopaminergic mechanism.

Atipamezole is a potent alpha2-adrenoceptor antagonist. Unlike various other alpha2-adrenoceptor antagonists, it has negligible affinity for any other neurotransmitter receptors such as alpha1-adrenergic, dopaminergic, GABAergic, serotonergic (such as 5-HT_{1A}) etc. receptors, thus being also a selective alpha2-adrenoceptor antagonists. The specificity and selectivity of various other known alpha₂-adrenoceptor antagonist have been questioned. Yohimbine has affinity also to various other than noradrenergic receptors such as dopaminergic, 5-hydroxyhyptaminergic receptors and benzodiazepine receptors. Idazoxan and also various other alpha₂-adrenoceptor antagonists such as RX821002, (2-methoxy idazoxan), delequamine (RS15385), BRL 44408 and ARC 239 have affinity also on 5-hydroxytryptamine (5-HT) 5-HT_{1A} receptors or 5-HT1D receptors, thus being less alpha2-adrenoceptor/5-HT receptor selective than atipamezole. Atipamezole is a potent antagonist in all alpha2-adrenoceptor subtypes and has mainly an effect on release of central noradrenaline, but the nonselective compound, yohimbine is known to significantly stimulate also central dopamine transmission (Haapalinna, A., Viitamaa, T., MacDonald, E., Savola, J.-M., Tuomisto, L., Virtanen, R. & Heinonen, E. (1997). Evaluation of the effects of a specific α₂-adrenoceptor antagonist, atipamezole, on α₁- and α₂-adrenoceptor subtype binding, brain neurochemistry and behaviour in comparison with yohimbine. (Naunyn-Schmiedeberg's Arch Pharmacol, 356, 570-582.).

It has been published that the rats trained to discriminate the alpha2-adrenoceptor antagonist idazoxan did not generalize d-amphetamine to idazoxan (Sanger D. J. Discriminative stimulus effects of the α₂-adrenoceptor antagonist idazoxan. Psychopharmacology 99: 117-121, 1989) and the rats trained to discriminate d-amphetamine did not generalize alpha2-adrenoceptor antagonists idazoxan and yohimbine to amphetamine (Sanger, D. J. Behavioural effects of the α₂-adrenoceptor antagonists idazoxan and yohimbine in rats: comparisons with amphetamine. Psychopharmacology 96: 243-249, 1988.). Thus, it has been suggested that the psychostimulant behavioural properties of alpha2-adrenoceptor antagonists have little in common with those of amphetamine. Therefore, it is unlikely that selective alpha2-adrenoceptor antagonists, that do not have direct effects on dopaminergic receptors or dopamine uptake site and have neglible effects on central dopamine metabolism when compared with the effects of yohimbine, would be generalized to psychostimulants.

### BRIEF DESCRIPTION OF THE DRAWINGS

FIG. 1 shows the generalization test results (% of atipamezole-associated lever selection) of atipamezole s.c. and p.o. administered 30 minutes (s.c.) or 60 minutes (p.o.) before the start of the session in rats trained to discriminate the α₂-adrenoceptor antagonist atipamezole 1 mg/kg s.c. in a two-lever operant drug discrimination paradigm (drug vs. no-drug), n=9-18/group.

FIG. 2 shows the generalization test results (% of atipamezole-associated lever selection) of the psychostimulants amphetamine s.c. and cocaine i.p. given 30 minutes before the session in rats trained to discriminate the α₂-adrenoceptor antagonist atipamezole 1 mg/kg s.c. in a two-lever drug discrimination paradigm (drug vs. no-drug discrimination), n—8/group.

FIG. 3 shows the generalization test results (% of atipamezole-associated lever selection) of MPV-1730 (of 4-(2-ethyl-5-fluoro-2,3-dihydro-1H-inden-2-yl)-1H-imidazole) s.c. given 30 minutes before the session in rats trained to discriminate the α₂-adrenoceptor antagonist atipamezole 1 mg/kg s.c. in a two-lever drug discrimination paradigm (drug vs. no-drug discrimination), n—8/group.

FIG. 4 shows the generalization test results (% of atipamezole-associated lever selection) of desipramine i.p. given 30 minutes before the session in rats trained to discriminate the α₂-adrenoceptor antagonist atipamezole 1 mg/kg s.c. in a two-lever drug discrimination paradigm (drug vs. no-drug discrimination), n=7/group.

### DETAILED DESCRIPTION OF THE INVENTION

Applicants have surprisingly discovered that a selective alpha2-adrenoceptor antagonist, atipamezole (4-(2-ethyl-2,3-dihydro-1H-inden-2-yl)-1H-imidazole hydrochloride) produced cue can be substituted by the psychostimulants d-amphetamine and cocaine, but not by noradrenaline uptake inhibitor desipramine in rats. Thus, selective alpha2-adrenoceptor antagonists, such as atiparnezole, and their pharmacologically acceptable esters or salts, can be used for prevention and treatment of physical dependence and withdrawal symptoms caused by the subacute use (even after a binge of a few days) of psychostimulant such as, but not limited to; nicotine, cocaine, amphetamine, dextroamphetamine. L-amphetamine, methamphetamine, ecstacy, phencyclidine, phenmetratzine, methylphenidate, diethylpropion, pemoline, mazindol, (−) cathione, fenfluramine (and other amphetamine derivatives having substitutions in aromatic ring). Physical dependence related withdrawal symptoms occurring after abrupt cessation of psychostimulant includes, but are not limited to: depression, anxiety, hyperphagia, continued sleepiness, anhedonia, sexual dysfunction (especially decrease in libido), dysphoria, lethargy, general fatigue, shivering, shaling, restlessness, headache, inability to concentrate, decreased sensory sensitivity, apathy and usually lead craving for the psychostimulant and relapse.

Alpha2-adrenoceptor antagonists of the invention include, without limitation, atipamezole, efaroxan, and their analogs and pharmaceutically acceptable salts. 4-(2-ethyl-2,3-dihydro-1H-inden-2-yl)-1H-imidazole, known as atipamezole, and its pharmaceutically acceptable acid addition salts with inorganic and organic acids generally used for the purpose, are described in U.S. Pat. No. 4,689,339, which is incorporated herein by reference. The halogenated analogs of atipamezole, for example 4-(2-ethyl-5-fluoro-2,3-dihydro-1H-inden-2-yl)-1H-imidazole and 4-(2-ethyl-5,6-difluoro-2,3-dihydro-1H-inden-2-yl)-1H-imidazole and their pharmaceutically acceptable acid addition salts have been described in U.S. Pat. No. 5,498,623, which is incorporated herein by reference. Efaroxan, 2-(2-ethyl-2,3-dihydro-2-benzofuranyl)-4,5-dihydro-1H-imidazole, and its pharmaceutically acceptable acid addition salts, are described in U.S. Pat. No. 4,411,908, which is incorporated herein by reference.

The precise amount of the drug to be administered to a mammal according to the present invention is dependent on numerous factors known to one skilled in the art, such as the compound to be administered, the general condition of the patient, the condition to be treated, the desired duration of use, the type of mammal, the method and route of administration, etc. For example, for atipamezole, the usual daily dosage will be from 1 to 50 mg, and can be from 10 to 30 mg, divided in 1 to 4 individual doses. In another embodiment, the dose for atipamezole will be about 10 mg. Typical routes of administration include, without limitation, oral, transdermal, transmucosal, and parenteral routes.

The treatment or use of the selective alpha2-adrenoceptor antagonist can be started, for example, at the time of the discontinuation of the use the psychostimulant agent. When the dose of the psychostimulant drug is decreased gradually, the use of the alpha2-adrenoceptor antagonist can be started before total discontinuation of substituted psychostimulant agent i.e. the alpha2-adrenoceptor antagonist may also be given together with a low dose psychostimulant.

The compounds of the invention may be used in conjunction with at least one further alpha2-adrenoceptor antagonist or at least one compound that is used to ease patient's withdrawal from psychostimulants psychostimulant drugs, such as antidepressants, antipsychotics and anxiolytics.

The compounds of the invention are void of side effects connected to previously known effects of psychostimulants. For instance, they have minor effect at therapeutic doses on cardiovascular functions, do not cause hyperactivity, anorexia, hyperthermia, suspiciousness and paranoia, bruxism, headache, nausea and vomiting and dizziness usually seen with compounds having direct effects at least on dopaminergic and/or 5-hydroxytryptaminergic (5-HT) or receptors and/or uptake sites. Furthermore, they will not cause motor dysfunctions (dyskinesias, dystonia, rigidity), hallucinations, euphoric or psychotic effects usually seen with compounds having direct effects on dopaminergic receptors and/or uptake sites. Moreover, they do not cause dependence and/or abuse liability usually seen with compounds having direct effects on dopaminergic and/or 5-hydroxyryptaminergic (5-HT) receptors and/or uptake sites or on glutaminergic system.

The invention will be further clarified by the following example, which is intended to be purely exemplary of the invention, and should not be construed as limiting.

### EXAMPLE 1

The effects of psychostimulants; d-amphetamine, cocaine, an alpha2-adrenoceptor antagonist; MPV-1730 and noradrenaline uptake inhibitor; desipramine were studied in rats discriminating atipamezole.

Animals and Pre-Experimental Care

A total of 50 Sprague-Dawley male rats (B&K, Sweden) were used in the drug discrimination experiment at Orion Pharma, Turku, Finland. The rats were housed in solid bottom polypropylene cages with stainless steel mesh lids 5 rats per cage on a 12/12 hour light/dark cycle (lights on at 06.00 a.m.) under standard conditions in 21±1° C. temperature. Softwood granulated aspen was used as bedding and the rats had a restricted diet. During training period the rats were allowed food 6-15 g/day immediately after the session and during testing period they were allowed 12-15 g/day after the session. The rats adopted well to this feeding schedule and grew slowly but steadily. Rats were drug and experimentally naive at the start of the experiments. All experimentation was approved by the local laboratory animal care and experimentation committee. The animals were housed according to the recommendations of Declaration of Helsinki and DHEW pub. No (NIH) 85-23 entitled “Guide for the care and use of laboratory animals”.

Drugs

The drugs used were atipamezole HCl and MPV-1730 HCl (Orion Pharma, Finland), d-amphetamine sulphate (Sigma, USA), cocaine HCl (Tamro, Finland)) and desipramine HCl (Sigma, USA). All doses refer to respective salt forms. Drugs were diluted in sterile purified water (Aquasteril, Orion Pharma, Finland) and were prepared daily. Injections were given 30 min before the sessions. Saline (Natrosteril, Orion Pharma, Finland) was used in control administrations. Drugs were administered subcutaneously (s.c.) or intraperiotoneally (i.p.) in a volume of 1 ml/kg or perorally (p.o.) gy a gavage 10 ml/kg.

Apparatuses

In the atipamezole discrimination experiment five identical operant chambers enclosed in larger sound and light attenuating, fan ventilated enclosures were used. Each chamber was equipped with two identical levers on one wall. Between these levers was a food cup, where 45 mg reward pellets (F0021, Bio-Serv, Frenchtown, USA) could be presented. The whole operant system was purchased from Rhema-Labortechnik, Hofheim, Germany.

Discrimination Training with Atipamezole

The training to press levers for reward pellets was started with 50 rats with a continuous reinforcement (CRF) schedule, when both levers were active and later by changing daily the active lever. The schedule was gradually increased and changing the active lever as follows: FR-2, FR-4, FR-6 and FR-10. The duration of the session to this point was 30 min and thereafter the training sessions were reduced to 15 min. The discrimination training dose of atipamezole (1 mg/kg s.c.) was selected according to the effect of atipamezole (0, 0.1, 0.3, 1 and 3 mg/kg s.c.) on FR-10 responding when tested in the rats (session 30, data not shown). Thereafter, discrimination training was started with 46 rats. The discrimination training was done in following two week sequence: S-D-D-S-S and D-S-S-D-D (D=drug appropriate lever, S=saline lever) (Colpaert F., Niemegeers, C., Janssen, P. Theoretical and methodological considerations on drug discrimination learning. Psychopharmacologia (Berl.) 46: 169-177, 1976.). For half of the rats the right lever was a drug-appropriate lever and for the another half the left lever. During training only the drug- or saline-appropriate lever was active, but both levers were recorded. If a rat chose the correct lever during ten consecutive sessions and the total number of responses before the first reinforcer was 15 or less, the rat was accepted to the drug tests.

Discrimination Testing

When the rats had reached the criterion, they were tested twice a week (usually on Wednesdays and Fridays) with different drugs. The normal sequence of saline or atipamezole was continued on other days. The lever selection had to be correct on the preceding day of the drug test and on the next day after drug test in order to be approved in the results. In the testing day, the rat decided which lever was activated by pressing ten lever presses on either of the levers. The selected lever was activated during the rest of the session. The previous saline day was used as a predrug control value. Rats could decide whether the cue produced by a certain drug was more like the cue produced by saline or atipamezole by completing 10 presses on the appropriate lever. A saline group and in the interaction tests also atipamezole 1 mg/kg group was always included in the experiments with different drugs.

Results

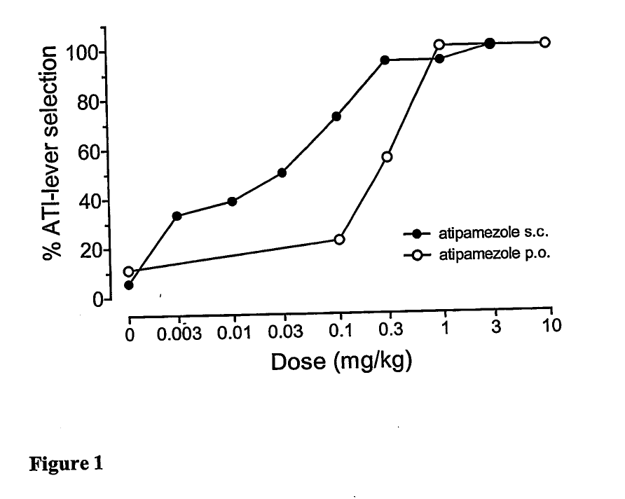

FIG. 1 illustrates the dose-response effect of the training drug atipamezole when the training dose was 1 mg/kg s.c. Atipamezole (0.003-10 mg/kg) dose-responsively increased atipamezole-associated lever selection. Full generalization was achieved at doses 0.3 mg/kg s.c. and 1 mg/kg p.o. These results show that the rats were able to discriminate atipamezole, i.e. could sense the central effect caused by atipamezole. Moreover, the rats were found to be very sensitive to atipamezole because low s.c. doses of atipamezole increased atipamezole lever selection.

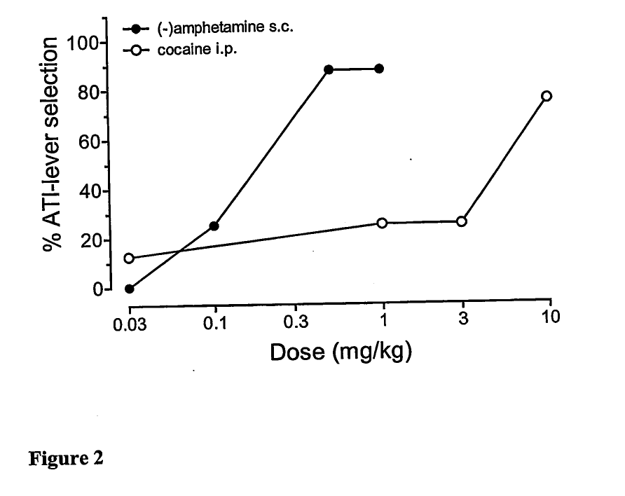

FIG. 2 indicates that d-amphetamine (0.03-1 mg/kg s.c.) was clearly generalized to atipamezole cue at doses 0.5 and 1 mg/kg. Cocaine (1-10 mg/kg i.p.) also produced almost total generalization to atipamezole cue at the dose of 10 mg/kg. These drugs thus showed an unexpected similarity in their discriminative abilities.

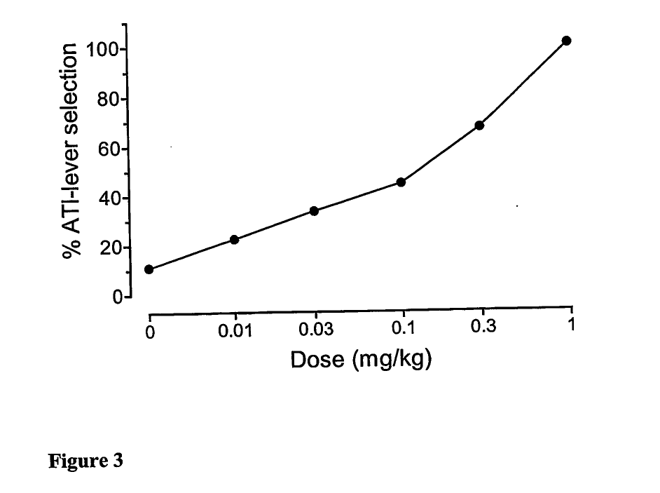

FIG. 3 shows the discrimination curve of other alpha2-adrenoceptor antagonist, MPV-1730 (0.01-1 mg/kg s.c.). MPV-1730 was generalized to atipamezole cue.

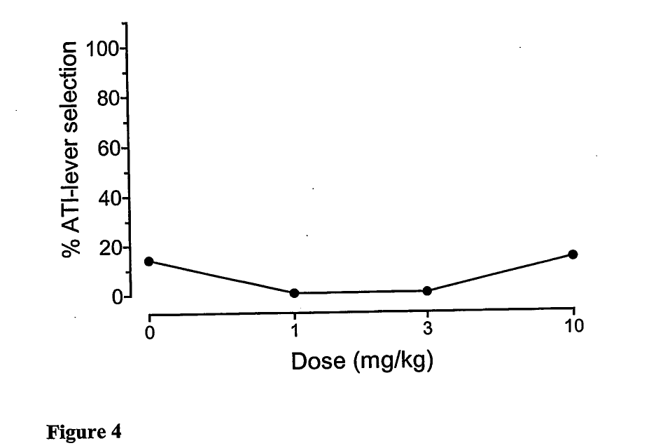

FIG. 4 shows that the noradrenaline uptake inhibitor desipramine is not generalized to atipamezole. The administration of higher doses of desipramine (20 and 30 mg/kg i.p.) had to be discontinued due to a prolonged lack of appetite. Thus, increasing the central noradrenaline tone by desipramine does not alone cause an effect similar to alpha2-adrenoceptor agonist atipamezole.

An example embodiment of the invention therefore includes a method for treating physical dependence and/or withdrawal symptoms caused by the discontinuation of the use of at least one psychostimulant agent, comprising administering to a mammal in need of said treatment at least one selective alpha2-adrenoceptor antagonist in an amount effective to ease the mammal's withdrawal from the psychostimulant. The mammal may be a human. The treatment may involve an effort to remedy or alleviate existing dependence and/or withdrawal symptoms. The treatment may also involve an effort to prevent withdrawal symptoms, for example, at the time of discontinuation of the use of a psychostimulant agent or any other time before withdrawal symptoms have developed.

The withdrawal symptoms may include, for example, depression, anxiety, hyperphagia, continued sleepiness, anhedonia, sexual dysfunction, dysphoria, lethargy, general fatigue, shivering, shaking, restlessness, headache, inability to concentrate, decreased sensory sensitivity and apathy.

Psychostimulant agents include amphetamine, dextroamphetamine, methamphetamine and other β-phenylisopropylamine derivatives. Example psychostimulant agents include cocaine, ecstacy, phencyclidine, phenmetratzine, methylphenidate, diethylpropion, pemoline, mazindol and (−) cathione. Psychostimulant agents can also generally include any compounds that enhance dopamine release and/or inhibit dopamine uptake from the synaptic cleft in the central nervous system.

Alpha2-adrenoceptor antagonists include atipamezole or a pharmaceutically acceptable salt thereof. Alpha2-adrenoceptor antagonists also include one or more of efaroxan and pharmaceutically acceptable salts thereof. Alpha2-adrenoceptor antagonists also include one or more of 4-(2-ethyl-5-fluoro-2,3-dihydro-1H-inden-2-yl)-1H-imidazole and pharmaceutically acceptable salts thereof. Alpha2-adrenoceptor antagonists also include at least one analog chosen from analogs of atipamezole and analogs of efaroxan. Alpha2-adrenoceptor antagonists further include at least one ester chosen from esters of atipamezole and esters of efaroxan.

The alpha2-adrenoceptor antagonist may be administered alone as an only active ingredient. The alpha2-adrenoceptor antagonist may also be administered with one or more other active ingredients, for example, with a low dose of psychostimulant. The alpha2-adrenoceptor antagonist may also be administered, for example, together with an antidepressant, antipsychotic or anxiolytic agent. The alpha2-adrenoceptor antagonist may also be administered to prevent relapse after withdrawal for psychostimulant.

1. A method for the treatment of physical dependence and/or one or more withdrawal symptoms caused by the discontinuation of the use of at least one psychostimulant agent in a mammal, which comprises administering to the mammal an effective amount of a selective alpha2-adrenoceptor antagonist.

2. A method as claimed in claim 1, which comprises treating the mammal for existing dependence and/or one or more existing withdrawal symptoms.

3. A method as claimed in claim 1, which comprises treating the mammal to prevent the development of dependence and/or one or more withdrawal symptoms.

4. A method as claimed in claim 1, which comprises discontinuing the use of the at least one psychostimulant upon administration of the at least one alpha2-adrenoceptor.

5. A method as claimed in claim 1, which comprises gradually reducing the use of the at least one psychostimulant ultimately to discontinuation while administering the at least one alpha2-adrenoceptor.

6. A method as claimed in claim 1, wherein the mammal is a human.

7. A method as claimed in claim 1, which comprises treating one or more withdrawal symptoms, wherein at least one withdrawal symptom is depression, anxiety, hyperphagia, continued sleepiness, anhedonia, sexual dysfunction, dysphoria, lethargy, general fatigue, shivering, shaking, restlessness, headache, inability to concentrate, decreased sensory sensitivity or apathy.

8. A method as claimed in claim 1, wherein the at least one psychostimulant agent is amphetamine, dextroamphetamine, methamphetamine or other β-phenylisopropylamine derivative.

9. A method as claimed in claim 1, wherein the at least one psychostimulant agent is nicotine, cocaine, ecstacy, phencyclidine, phenmetratzine, methylphenidate, diethylpropion, pemoline, mazindol, (−) cathione or fenfluramine.

10. A method as claimed in claim 1, wherein the alpha2-adrenoceptor antagonist is atipamezole or a pharmaceutically acceptable salt thereof.

11. A method as claimed in claim 1, wherein the alpha2-adrenoceptor antagonist is efaroxan or a pharmaceutically acceptable salt thereof.

12. A method as claimed in claim 1, wherein the at least one alpha2-adrenoceptor antagonist is 4-(2-ethyl-5-fluoro-2,3-dihydro-1H-inden-2-yl)-1H-imidazole or a pharmaceutically acceptable salt thereof.

13. A method as claimed in claim 1, which comprises further administering one or more antidepressants, antipsychotics or anxiolytic agents.

14. A method for the prevention of relapse after withdrawal from the use of at least one psychostimulant agent in a mammal, which comprises administering to the mammal an effective amount of a selective alpha2-adrenoceptor antagonist.

15. A method for the treatment of physical dependence and/or one or more withdrawal symptoms caused by the discontinuation of the use of at least one compound that enhances dopamine release and/or inhibits dopamine uptake from the synaptic cleft in the central nervous system, which comprises administering to the mammal an effective amount of a selective alpha2-adrenoceptor antagonist.
